(19) United States
(12) Patent Application Publication
(10) Pub. No.: US 2015/0320997 A1
(43) Pub. Date: Nov. 12, 2015

(54) Instrument for and method of treating venomous bites

(71) Applicant: Ottfried Waldenburg, Tucson, AZ (US)

(72) Inventor: Ottfried Waldenburg, Tucson, AZ (US)

(21) Appl. No.: 14/671,835

(22) Filed: Mar. 27, 2015

Related U.S. Application Data
(63) Continuation-in-part of application No. 13/839,057, filed on Mar. 15, 2013, now abandoned.

(51) Int. Cl. A61N 1/18; A61N 1/04; A61N 1/20

(52) U.S. Cl. CPC A61N 1/18; A61N 1/205; A61N 1/0468

(57) ABSTRACT

A method and instrument for treating the toxic effect a patient experiences as a result of insect bites and snakebites comprises applying multiple instantaneous 20,000 to 30,000 volt electric discharges from a hand-operated piezoelectric device to a site of the bite.

### RELATED PATENT APPLICATIONS & INCORPORATION BY REFERENCE

This utility application is a continuation-in-part application of U.S. application Ser. No. 13/839,057, entitled “METHOD OF TREATING VENOMOUS BITES,” filed Mar. 15, 2013. This related application is incorporated herein by reference and made a part of this application, including any and all U.S. patents, U.S. patent applications, and other documents, hard copy or electronic, cited or referred to during the prosecution this related application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The word “piezoelectricity” means electricity resulting from pressure.

### BACKGROUND

Venomous bites from insects and snakes may be treated by an electrical discharge as discussed in U.S. Pat. No. 5,792,211 by Teague.

### SUMMARY

My method and instrument are an improvement over Teague, and have one or more of the features depicted in the embodiment discussed in the section entitled “DETAILED DESCRIPTION OF ONE ILLUSTRATIVE EMBODIMENT.” These features are not listed in any rank order nor is this list intended to be exhaustive. The claims that follow define my instrument and method of treating venomous bites, distinguishing them from the prior art; however, without limiting the scope of my instrument and method as expressed by these claims, in general terms, some, but not necessarily all, of their features are:

I have discovered that piezoelectricity may be used to treat successfully the toxic effect a patient experiences as a result of a venomous insect bite or a venomous snakebite by applying to a site of such a bite an instantaneous electric discharge from a hand-operated piezoelectric instrument that is isolated from ground during use.

The instrument includes a housing, a barrel, and a handle sized and shaped to be gripped with one hand of a user. The housing contains a piezoelectric crystal held between two sections of a vice member. The barrel has a wire that extends lengthwise along the barrel and terminates at a tip that projects outward from the barrel. A trigger is positioned to be actuated by the index finger of a user while gripping the handle.

Thus, while a user holds the instrument by the handle with one hand and while gripping the trigger with an index finger of the one hand, depressing the trigger to actuate the mechanical linkage so that the two sections of the vice member apply pressure to the crystal to generate an electrical discharge at the tip, holding the tip directly on the site as the trigger is depressed.

### DESCRIPTION OF THE DRAWING

My method and instrument are discussed in detail in connection with the accompanying drawing, which is for illustrative purposes only. This drawing includes the following FIGURE (Fig.):

FIG. 1 is a schematic diagram of the instrument used in performing my method.

### DETAILED DESCRIPTION OF ONE ILLUSTRATIVE EMBODIMENT

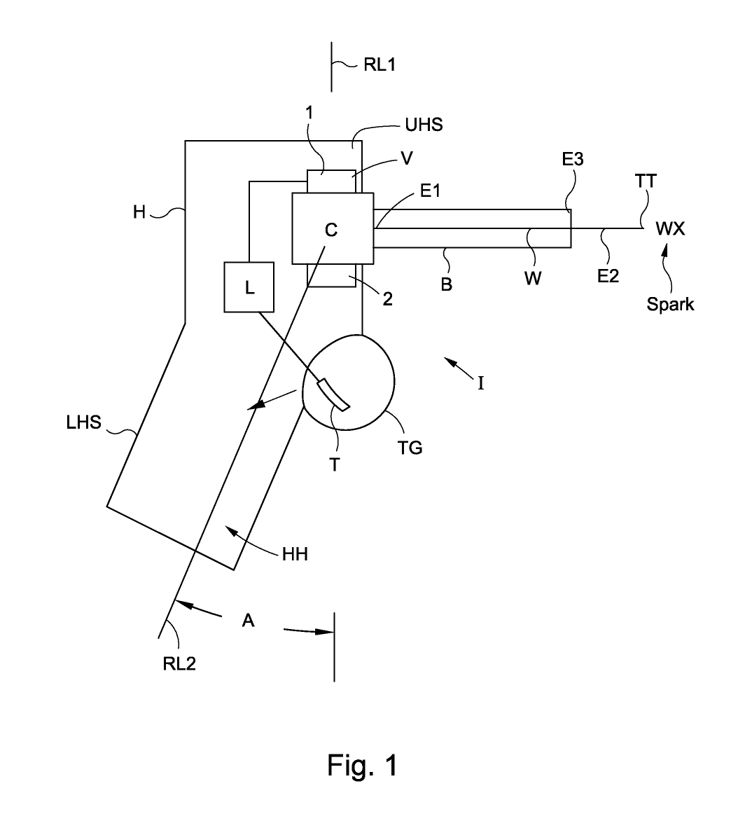

My method treats the toxic effect a patient experiences as a result of an insect bite or a snakebite. It comprises applying to a site of the bite an instantaneous electric discharge, a spark. A hand-operated piezoelectric instrument I such as illustrated in FIG. 1 is used in performing my method. Such an instrument I upon actuation generates a spark discharge at approximately of 20,000 to 30,000 volts, or greater. The voltage, however, is sufficiently low to avoid any injury to the patient. The instrument I is sized to be hand held, manually operated, and electrically isolated from ground during use.

As depicted in FIG. 1, the instrument I includes a housing H having within it a piezoelectric crystal C held between two sections 1 and 2 of a vice V. The housing has an upper housing section UHS and a lower housing Section LHS, with the lower housing section configured as a handle HH that can be grasped by a user with one hand while being used to actuate a trigger T. A piezoelectric crystal C is within the upper housing section UHS. Extending at a right angle to a longitudinal reference line RL1 of the housing H is an elongated barrel B with a central, axial, electrically conductive wire W that extends lengthwise along the length of the barrel B. The respective relative positions of the handle HH and barrel may be defined by an angle A between the reference line RF1 and a reference line RF2 that intersects with the barrel B. For example, this angle A may be from 0 to 90 degrees.

One end E1 of the wire W is in electrical contact with the crystal C and the other end E2 of the wire projects from of the end E3 of the barrel. By actuating the trigger T, at the tip TT of the wire W a spark is created as current discharges therefrom and is applied directly to the site of the bite. The trigger T is located along an inner side of the handle HH at a position so the user, in the conventional manner of gripping a hand held pistol, can actuate the trigger by pulling the trigger with the index finger while simultaneously holding the handle. A trigger guard TG at least partially surrounds the trigger T, but allows the index finger to be wrapped around the trigger while the instrument I is being held. A mechanical linkage L operably connects the trigger T to the vice V. Upon manually actuating the trigger T by depressing it, the two sections 1 and 2 of the vice V apply pressure to the crystal C. This pressure, typically 2-3 pounds, causes the crystal C to discharge a current along the wire W. At the tip TT of the wire W a spark is created as current discharges. The handle HH and trigger T are made of material and connected in a manner that the instrument is electrically isolated from ground during use.

The instrument I is a convenient and simple tool to use in practicing my method. To practice my method, the site of the bite is first cleaned. While holding the handle HH with one hand and gripping the trigger T with an index finger, the tip TT of the wire W from which the spark is emitted is held directly on the site of the bite. The trigger T of the instrument I is manually actuated by the user to release a spark directly to the bite and the area surrounding the bite. Multiple instantaneous electric discharges are applied to a site of a bite spaced apart by approximately from 20 to 40 seconds. For example, approximately 8-15 treatment discharges are effect in most cases.

### SCOPE OF THE INVENTION

The above presents a description of the best mode I contemplate of carrying out my method, and of the manner and process of making and using it, in such full, clear, concise, and exact terms as to enable a person skilled in the art to make and use. My method is, however, susceptible to modifications and alternate constructions from the illustrative embodiment discussed above which are fully equivalent. Consequently, it is not the intention to limit my method to the particular embodiment disclosed. On the contrary, my intention is to cover all modifications and alternate constructions coming within the spirit and scope of my method as generally expressed by the following claims, which particularly point out and distinctly claim the subject matter of my invention:

1. A method of treating a toxic effect a patient experiences as a result of an insect bite or snakebite, said method comprising the steps of
applying to a site of such a bite an instantaneous electric discharge from a hand-operated piezoelectric instrument that is isolated from ground during use,
said instrument including
a housing having a barrel and a handle,
said barrel including a wire that extends along the barrel and terminates at a tip that projects outward from the barrel,
said handle including a trigger,
said housing containing a piezoelectric crystal held between two sections of a vice member,
a mechanical linkage connecting the trigger to the vice member,
while a user holds the instrument by the handle with one hand and while gripping the trigger with a finger of said one hand, depressing said trigger to actuate the mechanical linkage so that the two sections of the vice member apply pressure to the crystal to generate the discharge at the tip,
holding the tip directly on the site as the trigger is depressed.

2. The method of claim 1 where each instantaneous electric discharge is at a voltage from 20,000 to 30,000 volts.

3. The method of claim 2 where said treatment discharges are spaced apart by approximately from 20 to 40 seconds.

4. The method of claim 3 where from 2 to 3 pounds of pressure is applied by the vice member to the crystal.

5. An instrument including
a housing,
a barrel and
a handle,
said barrel including a wire that extends along the barrel and terminates at a tip that projects outward from the barrel,
said handle including a trigger,
said housing containing a piezoelectric crystal held between two sections of a vice member,
a mechanical linkage connecting the trigger to the vice member,
said handle and trigger being electrically isolated from ground.
